(19) United States
(12) Patent Application Publication
(10) Pub. No.: US 2026/0306799 A1
(43) Pub. Date: Oct. 8, 2026

(54) MEDICAL TUBING PROTECTION SYSTEM

(71) Applicant: Casandra Bradley, Spanish Fork, UT (US)

(72) Inventor: Casandra Bradley, Spanish Fork, UT (US)

(21) Appl. No.: 19/169,109

(22) Filed: Apr. 3, 2025

(51) Int. Cl. A61M 25/02

(52) U.S. Cl. CPC A61M 25/02; A61M 2025/0206; A61M 2025/028; A61M 2205/0205

(57) ABSTRACT

A medical tubing protection system includes a belt, a shield, and a sleeve. The belt is designed to be worn around a body portion of a patient. The belt includes a belt aperture therein to be positioned over an insertion area where medical tubing is inserted into the body portion of the patient. The shield is designed to be coupled to the belt over the belt aperture to protect the medical tubing and prevent access to the medical tubing at the insertion area. The shield is designed to permit the medical tubing to pass between the shield and the belt for connection to a medical device. The sleeve is designed to be placed around the medical tubing between the belt and the medical device to prevent mishandling or damaging of the medical tubing. The sleeve includes a resilient material layer for cushioning and protection of the medical tubing.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to medical tubing devices and more particularly pertains to a new medical tubing protection system for protecting medical tubes against tugging, strangulation, kinking, coiling, and damage from the environment and other sources. Many people need to have medical tubes inserted in their bodies. It is important that the medical tubes be protected from twisting or tugging that can result in damage or inadvertent removal or inoperability of the medical tubes. With most patients, but especially children and infants, there is a risk of the patients voluntarily or accidentally removing or damaging the medical tubes. The new medical tubing protection system includes a belt with a shield to protect the medical tubes and related equipment at the insertion area, and also includes a sleeve to protect the exposed medical tubing between the patient and any medical devices or equipment.

### (2) Description of Related Art Including Information Disclosed Under 37 CFR 1.97 and 1.98

The prior art relates to medical tubing devices. The prior art, as best understood, does not disclose a medical tubing protection system that includes a belt, a shield, and a sleeve, wherein the shield is designed to be coupled to the belt over a belt aperture to protect medical tubing and prevent access to the medical tubing at an insertion area, and wherein the sleeve is designed to be placed around the medical tubing between the belt and the medical device to prevent mishandling or damaging of the medical tubing.

### BRIEF SUMMARY OF THE INVENTION

An embodiment of the disclosure meets the needs presented above in a medical tubing protection system generally comprising a belt, a shield, and a sleeve. The belt is designed to be worn around a body portion of a patient. The belt includes a belt aperture therein to be positioned over an insertion area where medical tubing is inserted into the body portion of the patient. The shield is designed to be coupled to the belt over the belt aperture to protect the medical tubing and prevent access to the medical tubing at the insertion area. The shield is designed to permit the medical tubing to pass between the shield and the belt for connection to a medical device. The sleeve is designed to be placed around the medical tubing between the belt and the medical device to prevent mishandling or damaging of the medical tubing. The sleeve includes a resilient material layer for cushioning and protection of the medical tubing.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a perspective view of a medical tubing protection system according to an embodiment of the disclosure.

FIG. 2 is a front perspective view of a component of an embodiment of the disclosure.

FIG. 3 is a rear perspective view of a component of an embodiment of the disclosure.

FIG. 4 is a front perspective view of a component of an embodiment of the disclosure.

FIG. 5 is a top perspective view of a component of an embodiment of the disclosure in use.

FIG. 6 is a top perspective view of a component of an embodiment of the disclosure in use.

FIG. 7 is a cross-sectional view of a component of the embodiment of the disclosure in FIG. 6 in use.

FIG. 8 is a perspective view of a component of an embodiment of the disclosure.

FIG. 9 is a perspective view of an embodiment of the disclosure.

FIG. 10 is a side view of an embodiment of the disclosure in use.

### DETAILED DESCRIPTION OF THE INVENTION

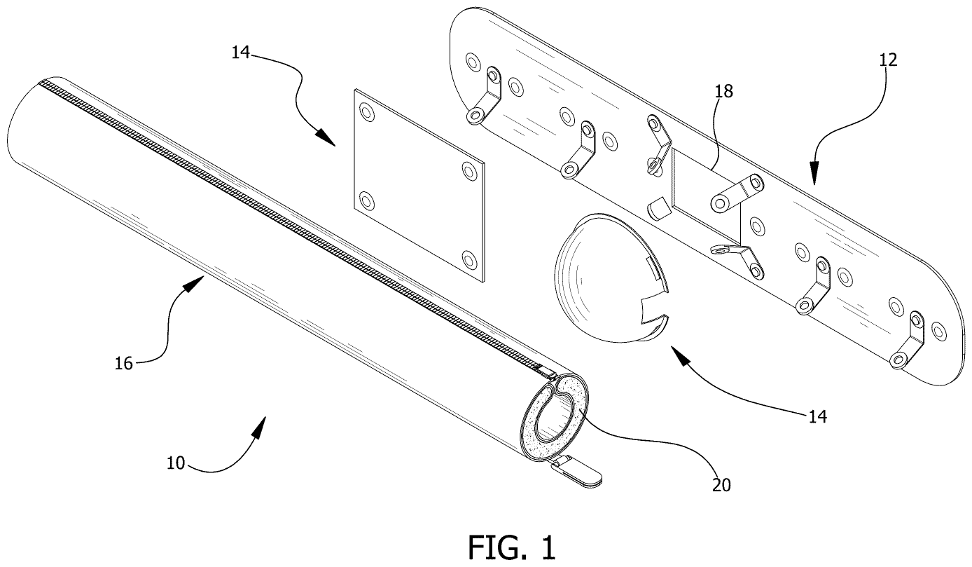

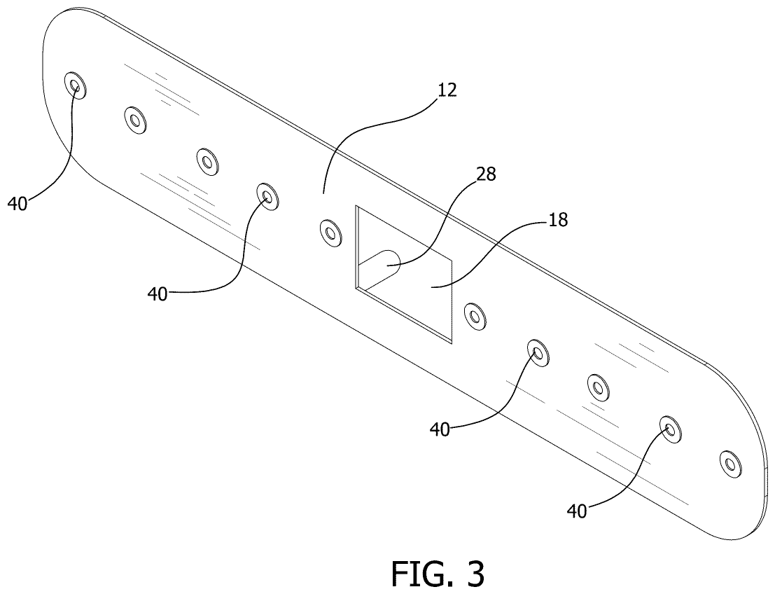

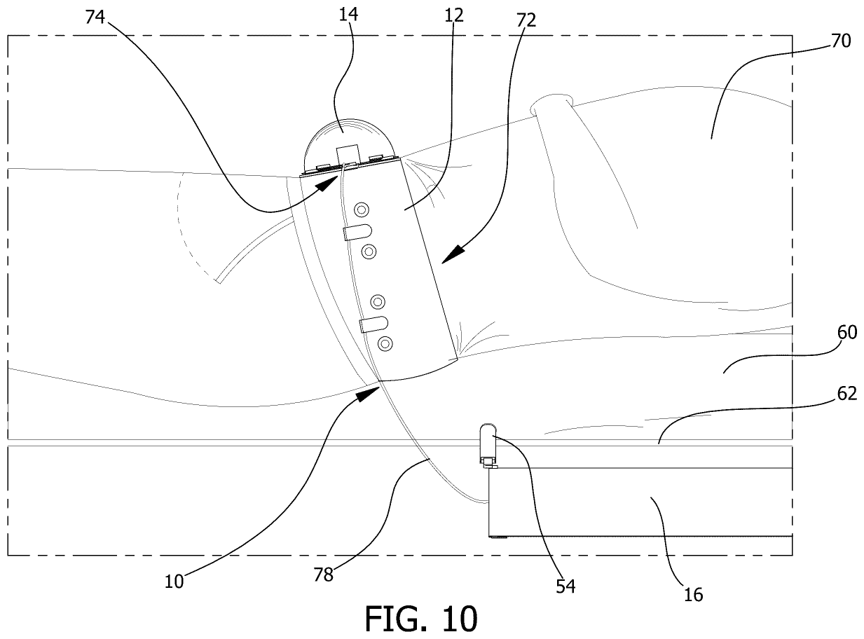

With reference now to the drawings, and in particular to FIGS. 1 through 10 thereof, a new medical tubing protection system embodying the principles and concepts of an embodiment of the disclosure and generally designated by the reference numeral 10 will be described.

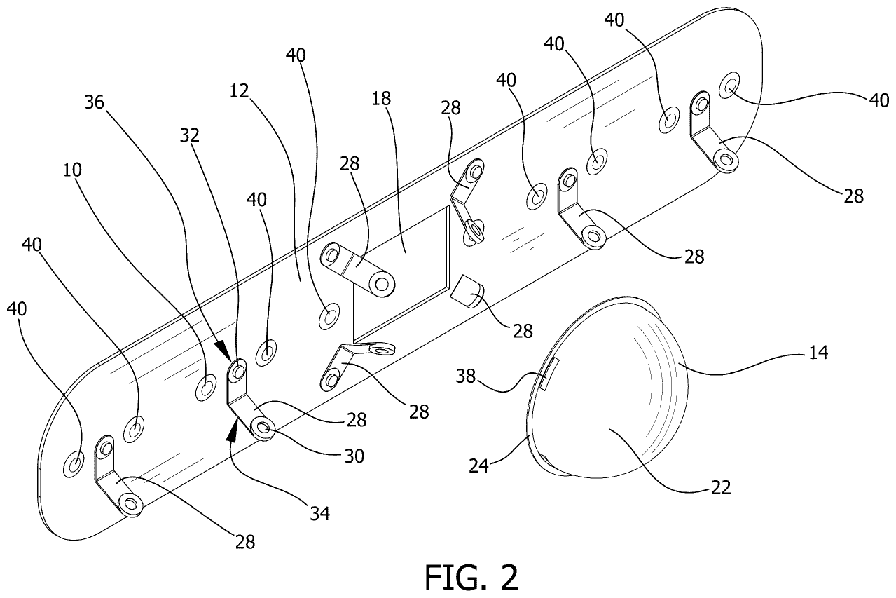

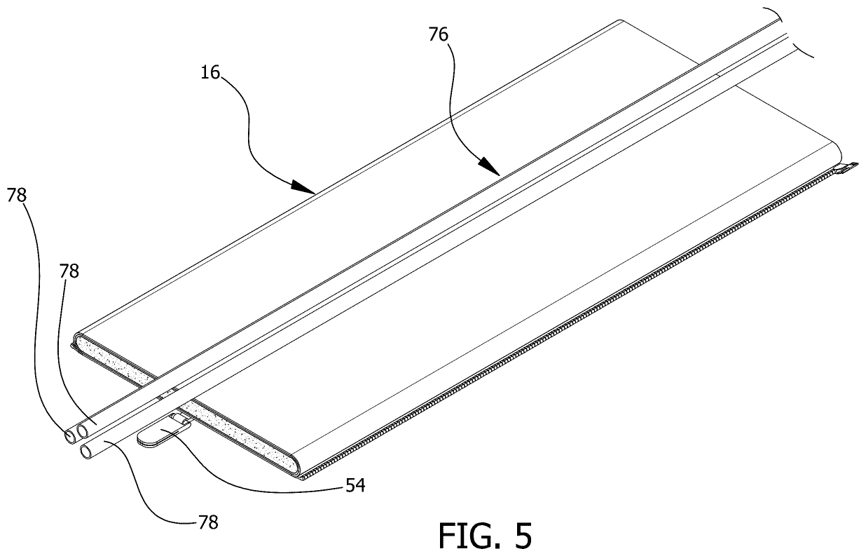

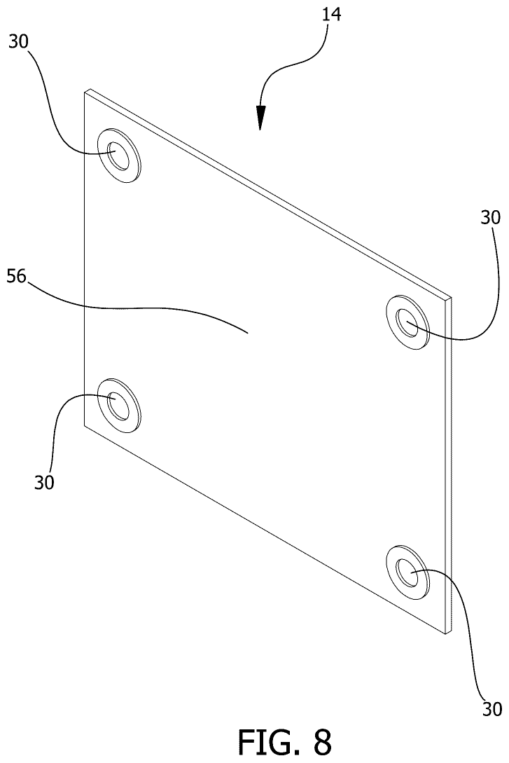

As best illustrated in FIGS. 1 through 10, the medical tubing protection system 10 generally comprises a belt 12, a shield 14, and a sleeve 16. The belt 12 is designed to be worn around a body portion 72 of a patient 70, as shown in FIG. 8. The belt 12 includes a belt aperture 18 therein to be positioned over an insertion area 74 where medical tubing 76 is inserted into the body portion 72 of the patient 70. As shown in FIGS. 2 through 4, the shield 14 is designed to be coupled to the belt 12 over the belt aperture 18 to protect the medical tubing 76 and prevent access to the medical tubing 76 at the insertion area 74. The shield 14 is designed to permit the medical tubing 76 to pass between the shield 14 and the belt 12 for connection to a medical device (not shown). As shown in FIGS. 5 through 7, the sleeve 16 is designed to be placed around the medical tubing 76 between the belt 12 and the medical device to prevent mishandling or damaging of the medical tubing 76. The sleeve 16 includes a resilient material layer 20 for cushioning and protection of the medical tubing 76.

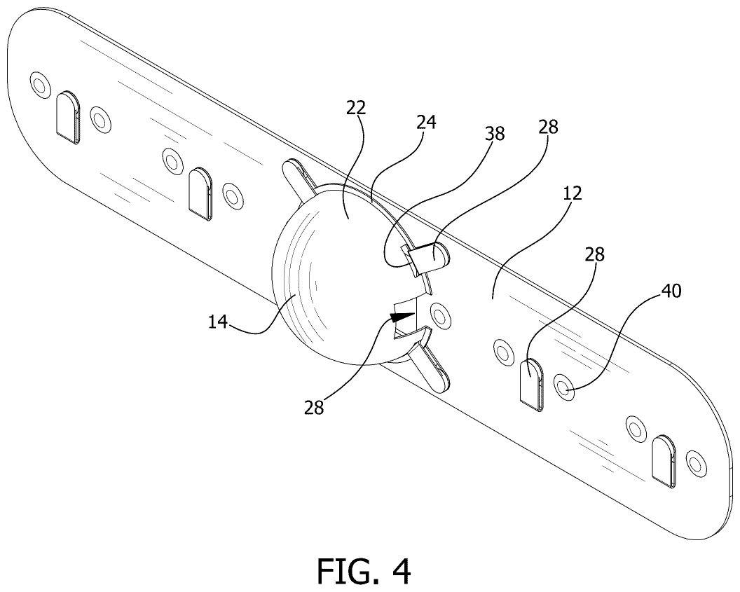

In the exemplary embodiment in FIGS. 2 through 4, the shield 14 is designed to project outwardly from the belt 12 such that a chamber is formed under the shield 14 to accommodate the medical tubing 76 and medical equipment positioned at the insertion area 74. The shield 14 is in the shape of a dome 22 designed to be placed against the outer surface of the belt 12. The shield 14 is spherical, though in another possible embodiment the shield 14 could have another shape, such as square, rectangular, or ovular. In the exemplary embodiment in FIG. 2, the shield 14 includes an annular flange 24 projecting from the dome 22, though this could be omitted in another possible embodiment. The shield 14 includes a shield orifice 26. The shield orifice 26 is designed for passage of the medical tubing 76 through the shield 14. In the exemplary embodiment in FIG. 4, the shield orifice 26 is a rectangular recess extending into an edge of the shield 14, though the shield orifice 26 could have essentially any shape and be positioned elsewhere in the shield 14. The shield orifice 26 is relatively small and positioned to direct the medical tubing 76 along the length of the belt 12. The shield orifice 26 is designed to permit passage of a plurality of medical tubes 78 through the shield 14, as it is not uncommon for more than one medical tube 78 to be inserted in the body portion 72.

The medical tubing protection system 10 further includes a plurality of belt straps 28 coupled to the belt 12. Each of the belt straps 28 includes a first coupler member 30 at a first strap end 34 and a second coupler member 32 at a second strap end 36 coupled to the belt 12. The first strap end 34 is designed to be coupled to the second strap end 36 such that each of the belt straps 28 is foldable into a loop to secure objects to the belt 12. In the exemplary embodiment in the figures, the belt straps 28 are made of a resilient material, such as an elastic fabric or similar, but could also be made of other material, such as textiles or plastics. The first coupler member 30 and the second coupler member 32 are in the form of snaps that are detachable from one another, though any other connecting structure, such as buttons or clasps, could be used as is well known in the mechanical arts.

As shown in FIGS. 2 and 4, the shield 14 includes a plurality of slots 38 spaced about a perimeter of the shield 14, such as by the annular flange 24. A set of the belt straps 28, specifically four in the exemplary embodiment, is positioned about the belt aperture 18. The first strap end 34 of each of the belt straps 28 positioned about the belt aperture 18 is designed to be inserted through a respective one of the slots 38 and then coupled to the second strap end 36 to form a loop to secure the shield 14 to the belt 12. It should be understood that the strap-and-slot connection of the shield 14 to the belt 12 is only one possible embodiment, as other connections, such as snaps or clasps, or possibly projecting tabs on the shield 14 that are insertable into pockets in the belt 12, could be utilized. In another possible embodiment, the shield 14 could be adhered or otherwise permanently attached to the belt 12.

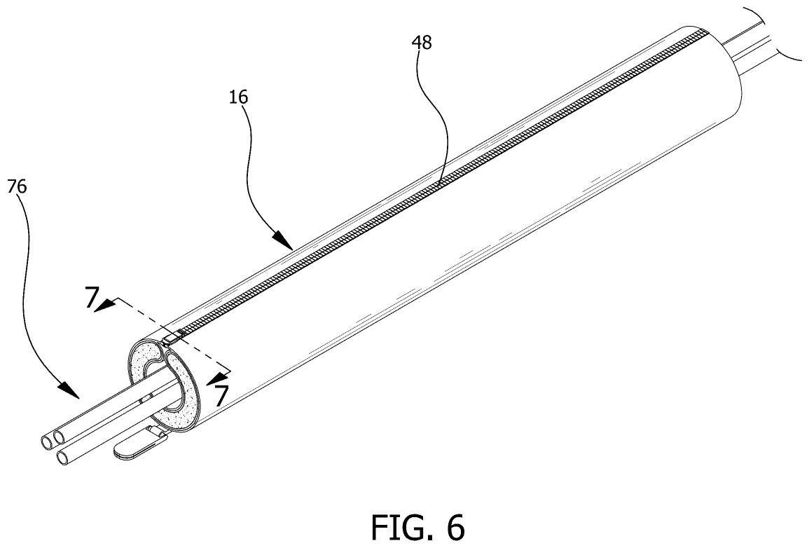

Another set of the belt straps 28 is positioned at intervals along the length of the belt 12. The first strap end 34 of the belt straps 28 along the length of the belt 12 is designed to be wrapped around the medical tubing 76 and then coupled to the second strap end 36 to form a loop to secure the medical tubing 76 to the belt 12. In the exemplary embodiment in FIGS. 4 and 6, each of the belt straps 28 has the same design. However, the design of the belt straps 28 could be different in another possible embodiment.

The medical tubing protection system 10 further includes a plurality of connectors 40 positioned along the length of the belt 12. Each of the connectors 40 is designed to connect opposing ends of the belt 12 to selectively adjust the length of the belt 12 to fit the body portion 72 of the patient 70. As is well known in the belt arts, belts can be made adjustable by a series of snaps, clasps, or holes designed to interact with other snaps, clasps, or buckles. In the exemplary embodiment in FIGS. 4 through 6, the connectors 40 are a series of snaps on an outer surface and an inner surface of the belt 12. To secure the belt 12 around the patient 70, the ends of the belt 12 can be overlapped and the front of the snaps inserted in a form or snap fit into the backs of the other snaps. However, essentially any known design of the connectors 40 could be used such that the belt 12 is adjustable for comfort and fit on the patient 70. In the exemplary embodiment, the belt 12 includes resilient material, such as elastic or stretchable fabric or plastic, such that the belt 12 is resiliently stretchable. In another possible embodiment, the belt 12 is made of non-resilient but flexible material.

In the exemplary embodiment in FIGS. 5 through 7, the sleeve 16 is designed to hold a plurality of medical tubes 78 therein, such as three medical tubes 78 as shown. The sleeve 16 could be completely closed such that the medical tubes 78 would have to be inserted by sliding or threading the medical tubes 78 through the sleeve 16. However, in the exemplary embodiment, the sleeve 16 includes a longitudinal gap 42 between a first sleeve edge 44 detachably coupled to a second sleeve edge 46, such that the sleeve 16 is designed to be selectively unrolled into a flat panel shape and rolled into a tube shape around the medical tubing 76, as shown in FIGS. 5 and 6.

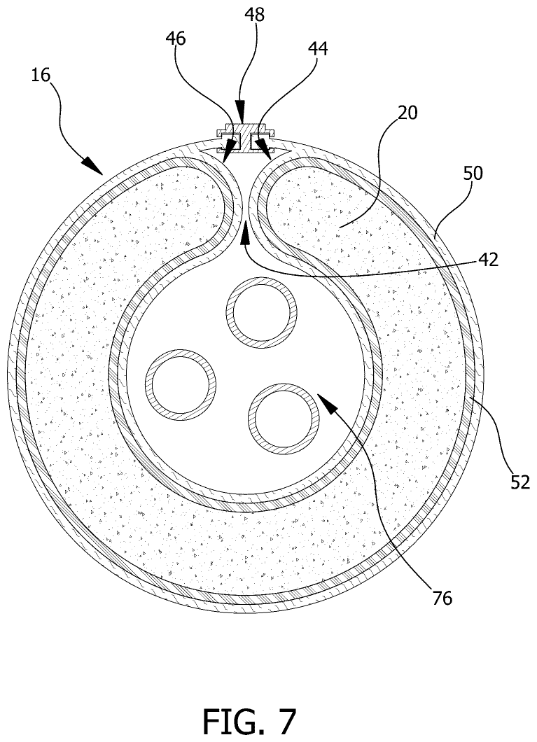

The sleeve 16 includes a zipper 48 designed to detachably couple the first sleeve edge 44 to the second sleeve edge 46, though other connections such as snaps or clasps or hook-and-loop material could be used. In the exemplary embodiment in FIG. 7, the sleeve 16 includes a fabric outer layer 50 surrounding the resilient material layer 20. The zipper 48 is connected to the fabric outer layer 50. In addition, the sleeve 16 includes an anti-microbial layer 52 positioned between the fabric outer layer 50 and the resilient material layer 20 and surrounding the resilient material layer 20. In the exemplary embodiment, the resilient material layer 20 consists of a foam and the anti-microbial layer 52 is vinyl. In one possible embodiment, the fabric outer layer 50 could be omitted and only the anti-microbial layer 52 included. In another possible embodiment, the anti-microbial layer 52 could be omitted and only the fabric outer layer 50 included.

In the exemplary embodiment in FIGS. 5 through 7, the sleeve 16 includes a sleeve coupler 54 to couple the sleeve 16 to a support structure, as shown in FIG. 8. In FIG. 8, the sleeve coupler 54 is coupled to a rail or structural portion 62 of a gurney or hospital bed 60, though the sleeve coupler 54 could easily be connected to other structures or objects, such as bedding, articles of clothing, or furniture. In the exemplary embodiment, the sleeve coupler 54 is in the form of a clip, though other known connectors could be used, such as strips of hook-and-loop material or elastic bands.

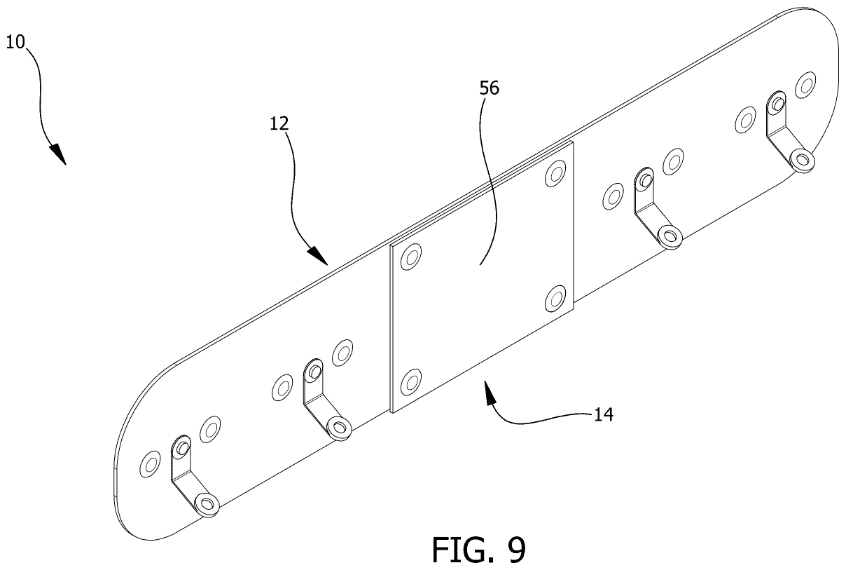

In another possible embodiment in FIGS. 8 and 9, the shield 14 could be a flat panel 56 that is coupled to the belt 12. In this embodiment, the shield 14 could be made of flexible material, such as cloth or fabric, or resiliently flexible and/or stretchable material, such as plastic or foam or elastic, or rigid or semi-rigid material, such as metal or plastic. In the exemplary embodiment in FIG. 9, the shield 14 in the form of the flat panel 56 is made of flexible elastic material, such that the medical tubing 76 could be slid in between the belt 12 and the shield 14. In another possible embodiment, the flat panel 56 could include the shield orifice 26 in the form of a hole in the flat panel 56 or a raised edge portion with space in between the flat panel 56 and the belt 12. The shield 14 could be fully detachable from the belt 12, or could be coupled along one edge and pivotable away from the belt, or permanently attached to the belt 12 on all edges. In the exemplary embodiment in FIG. 9, the flat panel 56 is coupled at the four corners by the first coupler members 30 to the second coupler members 32 of the belt straps 28. In one possible embodiment the flat panel 56 is used instead of the dome 22 or vice versa, whereas in another possible embodiment the flat panel 56 and the dome 22 are part of single kit, wherein the flat panel 56 and dome 22 can be exchanged as needed or desired.

In use, the belt 12 is placed around the body portion 72 of the patient 70, which body portion 72 is a torso as shown in FIG. 8, for example, but could alternatively be a leg or an arm. The belt aperture 18 is aligned with the insertion area 74 where the medical tubing 76 is inserted into the patient 70, such as via a medical device or a stoma, i.e., a surgical opening in the abdomen that connects an internal organ to the skin. The medical tubing 76 can be standard tubing for conducting liquids, such as medicine or bodily fluids. The shield 14 is coupled to the belt 12 to cover the belt aperture 18. The medical tubing 76 is passed through the shield orifice 26 and then held in place by the belt straps 28 positioned along the length of the belt 12. For medical tubing 76 extending beyond the belt 12 to a medical device or medical structure, the sleeve 16 is placed around this medical tubing 76, as shown in FIGS. 5 through 7. The medical tubing 76, which can be one or more individual medical tubes 78, is now gathered and held in a relatively secure manner to minimize or prevent tugging, strangulation, kinking, coiling, and general damage from objects in the surrounding environment, or by the voluntary or involuntary actions of the patient 70 or other persons or animals.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A medical tubing protection system comprising:
a belt, the belt being configured to be worn around a body portion of a patient, the belt including a belt aperture therein to be positioned over an insertion area where medical tubing is inserted into the body portion of the patient;
a shield, the shield being configured to be coupled to the belt over the belt aperture to protect the medical tubing and prevent access to the medical tubing at the insertion area, the shield being configured to permit the medical tubing to pass between the shield and the belt for connection to a medical device; and
a sleeve, the sleeve being configured to be placed around the medical tubing between the belt and the medical device to prevent mishandling or damaging of the medical tubing, the sleeve including a resilient material layer for cushioning and protection of the medical tubing.

2. The medical tubing protection system of claim 1, wherein the shield is configured to project outwardly from the belt such that a chamber is formed under the shield to accommodate the medical tubing and medical equipment positioned at the insertion area.

3. The medical tubing protection system of claim 2, wherein:
the shield is in the shape of a dome configured to be placed against the outer surface of the belt;
the shield includes a shield orifice; and
the shield orifice is configured for passage of the medical tubing through the shield.

4. The medical tubing protection system of claim 3, wherein the shield orifice is configured to permit passage of a plurality of medical tubes through the shield.

5. The medical tubing protection system of claim 1, further comprising a plurality of belt straps coupled to the belt, each of the belt straps including a first coupler member at a first strap end and a second coupler member at a second strap end coupled to the belt, the first strap end being configured to be coupled to the second strap end such that each of the belt straps is foldable into a loop to secure objects to the belt.

6. The medical tubing protection system of claim 5, wherein:
the shield includes a plurality of slots spaced about a perimeter of the shield; and
a set of the belt straps is positioned about the belt aperture; and
the first strap end of each of the belt straps positioned about the belt aperture is configured to be inserted through a respective one of the slots and then coupled to the second strap end to form a loop to secure the shield to the belt.

7. The medical tubing protection system of claim 5, wherein:
a set of the belt straps is positioned at intervals along the length of the belt; and
the first strap end of each of the belt straps along the length of the belt is configured to be wrapped around the medical tubing and then coupled to the second strap end to form a loop to secure the medical tubing to the belt.

8. The medical tubing protection system of claim 1, further comprising a plurality of connectors positioned along the length of the belt, each of the connectors being configured to connect opposing ends of the belt to selectively adjust the length of the belt to fit the body portion of the patient.

9. The medical tubing protection system of claim 1, wherein the belt comprises resilient material such that the belt is resiliently stretchable.

10. The medical tubing protection system of claim 1, wherein the sleeve is configured to hold a plurality of medical tubes therein.

11. The medical tubing protection system of claim 1, wherein the sleeve includes a longitudinal gap between a first sleeve edge detachably coupled to a second sleeve edge, such that the sleeve is configured to be selectively unrolled into a flat panel shape and rolled into a tube shape around the medical tubing.

12. The medical tubing protection system of claim 11, wherein the sleeve includes a zipper configured to detachably couple the first sleeve edge to the second sleeve edge.

13. The medical tubing protection system of claim 1, wherein the sleeve includes a fabric outer layer surrounding the resilient material layer.

14. The medical tubing protection system of claim 1, wherein the sleeve includes an anti-microbial layer positioned between the fabric outer layer and the resilient material layer and surrounding the resilient material layer.

15. The medical tubing protection system of claim 1, wherein the resilient material layer consists of a foam.

16. The medical tubing protection system of claim 1, wherein the sleeve includes a sleeve coupler to couple the sleeve to a support structure.

17. The medical tubing protection system of claim 1, wherein the shield is a flat panel of flexible material.

18. The medical tubing protection system of claim 4, further comprising a plurality of belt straps coupled to the belt, each of the belt straps including a first coupler member at a first strap end and a second coupler member at a second strap end coupled to the belt, the first strap end being configured to be coupled to the second strap end such that each of the belt straps is foldable into a loop to secure objects to the belt.

19. The medical tubing protection system of claim 18, wherein:
the shield includes a plurality of slots spaced about a perimeter of the shield; and
a first set of the belt straps is positioned about the belt aperture;
the first strap end of each of the belt straps positioned about the belt aperture is configured to be inserted through a respective one of the slots and then coupled to the second strap end to form a loop to secure the shield to the belt;
a second set of the belt straps is positioned at intervals along the length of the belt; and
the first strap end of each of the belt straps along the length of the belt is configured to be wrapped around the medical tubing and then coupled to the second strap end to form a loop to secure the medical tubing to the belt.

20. The medical tubing protection system of claim 19, further comprising a plurality of connectors positioned along the length of the belt, each of the connectors being configured to connect opposing ends of the belt to selectively adjust the length of the belt to fit the body portion of the patient, and further comprising an additional shield in the form of a flat panel of flexible material selectively interchangeable with the dome, wherein:
the belt comprises resilient material such that the belt is resiliently stretchable;
the sleeve is configured to hold a plurality of medical tubes therein;
the sleeve includes a longitudinal gap between a first sleeve edge detachably coupled to a second sleeve edge, such that the sleeve is configured to be selectively unrolled into a flat panel shape and rolled into a tube shape around the medical tubing;
the sleeve includes a zipper configured to detachably couple the first sleeve edge to the second sleeve edge;
the sleeve includes a fabric outer layer surrounding the resilient material layer;
the sleeve includes an anti-microbial layer positioned between the fabric outer layer and the resilient material layer and surrounding the resilient material layer;
the resilient material layer consists of a foam; and
the sleeve includes a sleeve coupler to couple the sleeve to a support structure, which sleeve coupler is in the form of a clip.
